(19) United States
(12) Patent Application Publication
(10) Pub. No.: US 2026/0263171 A1
(43) Pub. Date: Sep. 10, 2026

(54) HAND ASSIGNMENT FOR A ROBOTIC SURGICAL SYSTEM

(71) Applicant: Auris Health, Inc., Santa Clara, CA (US)

(72) Inventor: Drake Long, Santa Clara, CA (US)

(21) Appl. No.: 19/663,168

(22) Filed: Apr. 29, 2026

Related U.S. Application Data
(63) Continuation of application No. PCT/IB2024/060895, filed on Nov. 4, 2024.
(60) Provisional application No. 63/595,670, filed on Nov. 2, 2023.

(51) Int. Cl. A61B 34/00; A61B 34/37

(52) U.S. Cl. CPC A61B 34/25; A61B 34/37; A61B 34/74

(57) ABSTRACT

Among other things, hand assignment interfaces are disclosed that allow a surgeon to manually assign instruments to their left or right hands in a robotic surgical system. A robotic surgical system can include a left hand interface device, a right hand interface device, a display, and a processor. The processor can be configured to: present, on the display, a graphical user interface for assigning instruments to the left hand interface device or the right hand interface device; and, upon a user interaction that assigns a first instrument to the left hand interface device or the right hand interface device, reassign a second instrument in accordance with predefined logic.

### PRIORITY

This application is a continuation of International Application No. PCT/IB2024/060895, filed Nov. 4, 2024, entitled “HAND ASSIGNMENT FOR A ROBOTIC SURGICAL SYSTEM,” which claims priority to U.S. Provisional Application No. 63/595,670, filed Nov. 2, 2023, entitled “DYNAMIC USER INTERFACE FOR ASSIGNING SURGICAL INSTRUMENTS AND ACCESSORIES TO THE SURGEON'S LEFT OR RIGHT HUMAN INTERFACE DEVICE (HID),” the disclosures of which are incorporated by reference herein, in their entirety.

### BACKGROUND

Minimally invasive medical procedures, such as endoscopy or robotically-assisted surgery, are increasingly used for the diagnosis or treatment of a variety of patient conditions. These techniques are attractive for their potential to minimize trauma to the patient, reduce recovery times, enhance surgeon precision, or facilitate new surgical approaches that may not be possible with traditional technologies. Minimally invasive procedures often involve insertion of elongate instruments into a patient's body through small anatomical openings, such as natural orifices or small incisions. These instruments are then advanced to an anatomical site and used to observe, manipulate, or interact with tissue or objects within the patient. A physician may control these instruments via teleoperation by inputting commands to a physician console.

### BRIEF DESCRIPTION OF DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts an example of a surgical system.

FIG. 2 depicts an example of a physician console that may be employed in the surgical system of FIG. 1.

FIG. 3 depicts an example of a viewer interface that may be employed in the physician console of FIG. 2.

FIG. 4 depicts an example of a hand assignment interface that may be employed in the physician console of FIG. 2.

FIG. 5A depicts an example of a hand assignment interface employing a handedness-centric approach.

FIG. 5B depicts an example of a viewer interface employing a handedness-centric approach in accordance with the hand assignment interface of FIG. 5A.

FIG. 6 depicts an example of a hand assignment interface employing a card-based approach to the handedness-centric interface of FIG. 5A.

FIG. 7A depicts an example of a drag and drop interaction with the card-based interface of FIG. 6, in a first state where a user presses and holds a card.

FIG. 7B depicts the drag and drop interaction of FIG. 7A, in a second state after the user has dragged the card to a desired slot.

FIG. 7C depicts the drag and drop interaction of FIG. 7A and FIG. 7B, in a third state after the user has released the card in the desired slot.

FIG. 8A depicts an example of a tap interaction with the card-based hand assignment interface of FIG. 6, in a first state where a user has tapped a card.

FIG. 8B depicts the tap interaction of FIG. 8A, in a second state where a user has tapped a desired slot for the card.

FIG. 8C depicts the tap interaction of FIG. 8A and FIG. 8B, in a third state after the user has tapped the desired slot for the card.

FIG. 9A depicts an example of a hand assignment interface that is dynamically updated in accordance with a state of a surgical robot, in a first state where no instruments are loaded.

FIG. 9B depicts an example of a hand assignment interface that is dynamically updated in accordance with a state of a surgical robot, in a second state where a scope is loaded.

FIG. 9C depicts an example of a hand assignment interface that is dynamically updated in accordance with a state of a surgical robot, in a third state where two instruments are loaded.

FIG. 9D depicts an example of a hand assignment interface that is dynamically updated in accordance with a state of a surgical robot, in a fourth state where three instruments are loaded.

FIG. 9E depicts an example of a hand assignment interface that is dynamically updated in accordance with a state of a surgical robot, in a fifth state where three instruments are loaded and a fourth instrument is removed from an arm that is docked.

FIG. 10 depicts an example of a hand assignment interface when a user attempts to reassign an instrument to a hand that is full.

FIG. 11A depicts an example of a hand assignment interface employing an arm-centric approach with a robot model.

FIG. 11B depicts an example of a viewer interface employing an arm-centric approach.

FIG. 12A depicts an example of a hand assignment interface of FIG. 11A at a first step, where an instrument is selected for reassignment by a user, and at a second step, where an assignment selection menu is presented to the user.

FIG. 12B depicts the hand assignment interface of FIG. 12A at a third step, after the user has selected a desired assignment for the instrument, and at a fourth step, after the user has confirmed the instrument reassignment.

FIG. 12C depicts an example of a viewer interface employing an arm-centric approach.

FIG. 13 depicts an example of a hand assignment interface employing an arm-centric approach without a robot model.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description and appended drawings contain certain examples and configurations of this technology and are not intended to be an exhaustive disclosure of the only configurations in which the technology may be practiced. Other examples, features, aspects, embodiments, and advantages of the technology will be apparent to those skill in the art from this disclosure. As will be realized, the technology described herein is capable of other different and obvious aspects, all without departing from the inventive concepts disclosed herein. Accordingly, the drawings and descriptions should be regarded as illustrative in nature and not restrictive. In some instances, well-known structures and components are not described in detail or are shown in block diagram form to avoid obscuring concepts of this technology.

In robotic surgical systems, a surgeon may be able to control multiple instruments (sometimes referred to herein as “tools”). For example, in a four-arm laparoscopic robot, a surgeon can control up to four instruments at a time. The surgeon can control the robotic arms with their hands using human interface devices (HIDs) (sometimes referred to herein as “haptic interface devices” or “input devices”) located on a surgeon console. In some instances, an algorithm can run to automatically assign tools to either be controlled by a surgeon's left HID or right HID, due to the complex architecture of the system. For example, the algorithm may run to optimize the system for use and/or to reduce collisions.

In some configurations, all tools can be assigned to a specific HID except the laparoscope (sometimes referred to herein as a “scope”), which can be controlled using both HIDs. The laparoscope can be classified as the central instrument so that instruments loaded on a robot arm to the left of the scope are assigned to the surgeon's left HID, and instruments loaded on a robotic arm to the right of the scope are assigned to the surgeon's right HID. If the algorithm fails to run, isn't confident in its results, assigns instruments incorrectly, or the surgeon wants more control, it may be beneficial to provide a surgeon with a way to manually assign instruments to their left or right HID. Surgeon's often refer to their HIDs as their hands, so in this description, HIDs will sometimes be referred to as the surgeon's hands.

Among other things, the following description relates to a hand assignment interface that can allow a surgeon to manually assign instruments to their left or right hands. In some configurations, the hand assignment interface displays on a touchscreen display of a surgeon console. These and other features of this technology are further described below with respect to examples shown in the figures. However, there are multiple inventive concepts disclosed herein which may be practiced independently, in combination, or in other contexts beyond these particular examples. Accordingly, these examples are explanatory in nature but should not be construed as limiting.

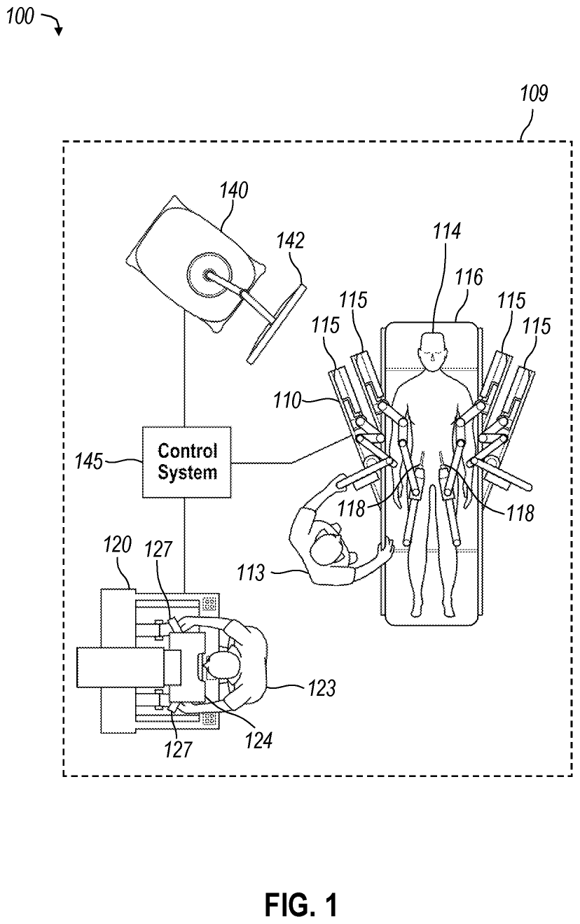

FIG. 1 depicts an example of a surgical system, in accordance with some embodiments. In the illustrated configuration, the surgical system 100 is configured as a robotic surgical system deployed for robotically-assisted surgery. The surgical system 100 can be used to perform a variety of surgical procedures to diagnose and/or treat a patient 114. Some examples of procedures include laparoscopy, endoscopy, bronchoscopy, thoracoscopy, urological procedures, vascular procedures, and/or gastrointestinal (GI) procedures.

As seen in FIG. 1, surgical system 100 includes a surgical robot 110, a physician console 120, and a support tower 140. These components are set up in procedure area 109, such as an operating room or an endoscopy suite, and may be used in concert with each other to perform a procedure on patient 114. The various components of the surgical system may be coupled physically, communicatively, and/or operatively as appropriate to facilitate operation of the surgical system 100.

Surgical robot 110 is configured to interact with patient 114 and perform various tasks based on commands received from physician console 120. Surgical robot 110 can include one or more robotic manipulators 115 configured to manipulate one or more instruments 118 (or “tools”) to perform various surgical tasks. These instruments 118 can be inserted into a body of patient 114 (e.g., through a laparoscopic incision, natural orifice, and/or port) to access an anatomical site and facilitate surgical tasks such as manipulating tissue or capturing endoscopic images with the tips of these instruments. In various configurations, the robotic manipulator(s) 115 may be configured to manipulate multiple different types of instruments in a procedure and/or across different procedures, allowing the manipulators to utilize such instruments to perform various surgical tasks. Some examples of surgical instruments manipulatable by the robot include graspers, forceps, scissors, scopes, hooks, needle drivers, staplers, biopsy tools, energy delivery instruments, suction devices, irrigation devices, and/or steerable catheters.

In some configurations, as seen in FIG. 1, surgical robot 110 can include multiple robotic manipulators 115 configured to manipulate multiple instruments 118. In the illustrated configuration, the surgical robot 110 includes four robotic manipulators 115, where each manipulator can manipulate a corresponding instrument 118. In some variations, surgical robot 110 can employ any suitable number of one or more robotic manipulators. For example, in various configurations the surgical robot 110 may include one, two, three, four, five, six, or more robotic manipulators, where each robotic manipulator is configured to manipulate one or multiples instruments. The robotic manipulators 115 can each include one or more actuators (e.g., motors) that can be electronically controlled to manipulate the instruments 118. For example, a robotic manipulator 115 can be actuated to control a position of an instrument 115 within the patient's body and/or to actuate mechanisms of the instrument (e.g., articulate or actuate an instrument tip in one or more degrees of freedom). Each robotic manipulator 115 can include, for example, a robotic arm having a series of links connected by a series of joints, where a distal end of the robotic arm is configured to couple with the corresponding instrument. Alternatively, or in combination, a robotic manipulator can include a carriage or motorized platform that may move along a track to control an instrument or interact with patient 114. In some instances, one or more users, such as surgical staff member 113, can mount or couple various instruments 118 to the various robotic manipulators 115 during initial set up and/or throughout a procedure to exchange instruments. As instruments are mounted to the various robotic manipulators 115, the robot 110 can be configured to detect presence and/or identify the corresponding instruments using sensing or identification technologies, such as optical sensing, magnetic sensing, radio frequency identification (RFID), or the like.

In the example shown in FIG. 1, surgical robot 110 is configured as a table-based system, where the robotic manipulators 115 are coupled to, or integrated with, the surgical table 116 that supports patient 114. In some variations, the surgical robot 110 can be configured as a robotic cart that can be positioned beside the patient 114 and/or beside the operating table 116. Additionally, or alternatively, the robot can be configured as a boom-based robot, where robotic manipulators descend from an overhead boom that can be suspended above the patient. In some variations, the surgical system 100 can include one or multiple robots 110 or robotic carts, where each robot or cart supports one or multiple robotic manipulators or robotic arms, and where the multiple robots or robotic carts are configured to operate in cooperation with each other.

Physician console 120 includes one or more input devices 127, which a user (e.g., physician 123) can operate to provide commands for teleoperation of robot 110. As illustrated, each input device 127 is configured as a handheld device that the physician 123 can manipulate with their hands to provide input to the system. In various configurations, the physician console 120 can employ one or several types of input devices to provide various modes for the physician 123 to interact with the surgical system 100. Examples of input devices include pendants, gimbal-based controllers, graspers, touch sensors, trackballs, joysticks, buttons, and/or foot pedals.

Physician console 120 can also include one or more displays 124, which can be configured to present images for observation by the physician 123. For example, display(s) 124 can be configured to display endoscopic images captured with the instruments 118, so that user can provide commands to the robot 110 via the input device(s) 127 while viewing a real-time camera feed captured within the patient's anatomy. Alternatively, or in combination, a display 133 can be configured to display, for example, pre-operative images, navigation information, or interactive menus. Examples of displays include flat panel displays, stereoscopic displays, head-mounted displays, liquid crystal displays (LCD), organic light emitting diode (OLED) displays, touch screen displays, and/or other types of electronic display devices. Physician console 106 can be configured to provide inputs or receive outputs from the robot 110 or the instruments 118 via cabling and/or wireless communication.

The support tower 140 can interact with surgical robot 110, instruments 118, and/or physician console 120 to provide various supporting functionality to the system, such as vision processing, fluidics, and/or energy generation. For example, the support tower 140 can provide vision processing, light generation, navigation support, fluidics, and/or energy generation for various instruments 118 or components of the surgical system 100. Alternatively, or in combination, support tower 140 can provide an interface for one or more users, such as surgical staff 113, to interact with the surgical system. In the illustrated example, support tower 140 includes a display 142 that can be configured to present any of the same information described herein with respect to the physician console and/or additional information.

In the illustrated example, surgical robot 110, physician console 120, and support tower 140 are illustrated as separate components that may be positioned in various locations in procedure area 109. In some variations, the surgical system 100 may embody any two or more of these components as integrated components. For example, in some configurations, the support tower 140 may be provided as an integral component of the physician console 120 or surgical robot 110.

Control system 145 includes processing circuitry and memory communicatively coupled to the robot 110, physician console 120, and/or support tower 140. Control system 145 can be configured to implement functions of the surgical system 100, such as controlling or actuating the robot 110, controlling or operating the instruments, or processing inputs or outputs to or from physician console 120. For example, processing circuitry of the control system 145 can be configured via hardware or software programming to implement any functions described further herein in connection with operation of surgical system 100. Examples of processing circuitry include one or more central processing units (CPUs), graphics processing units (GPUs), field programmable gate arrays (FPGAs), application specific integrated circuits (ASICs), or other processors configured to process inputs or outputs for the surgical system 100. As used herein, the term “processor” can encompass a single processing chip or integrated circuit, or multiple processing chips or integrated circuits that may be co-located or distributed in different locations and configured to execute functions described herein. Memory can store instructions that, when executed by the processor, cause execution of methods described herein. As used herein, the term “memory” can encompass any suitable non-transitory computer readable medium embodied in one or several memory devices, such as hard drives, flash memory, solid state memory, storage discs, or tapes. Components of the control system 145 may be physically located in, or physically connected to components of the surgical system 100, such as the robot 110 or the physician console 120, or components of the control system 145 may be otherwise communicatively coupled to components of surgical system 100 via various wired or wireless interconnections.

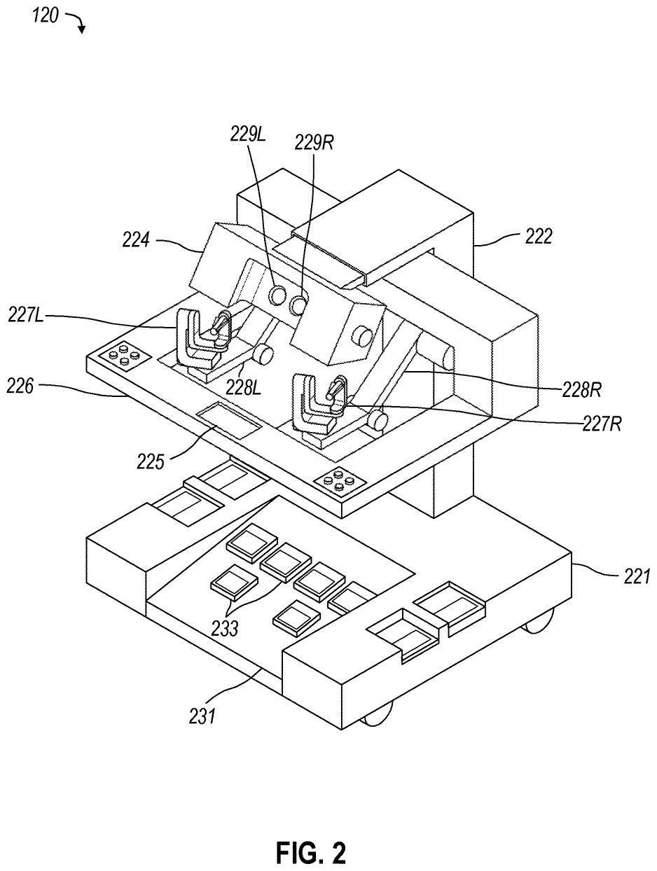

FIG. 2 depicts an example of the physician console 120 that may be employed in the surgical system of FIG. 1, in accordance with some embodiments. As noted above, physician console 120 (sometimes referred to herein as a “surgeon console”) can provide an interface for a physician (e.g., a surgeon) to interact with the surgical system 100, for example to control the surgical robot. In some instances, other users, such as surgical staff members may also interact with the physician console 120.

As seen in FIG. 2, physician console 120 can include a base 221, a pillar 222 (or column) coupled to the base 221, and a viewer display 224 coupled to the pillar 222. Alternatively, or in combination, the pillar 222 can support an armrest 226. The viewer display 224 can be supported by the base 221 via the pillar, and can provide a primary display for a physician to view endoscopic images. In the illustrated example, viewer display 224 is configured as an immersive, three-dimensional, stereoscopic display having a left eye display 229L and a right eye display 229R configured to present three-dimensional images to the physician when the physician inserts their head into the viewer housing. Although shown with an immersive design, in some variations, the physician console 120 may be provided with an open design, where the primary display is configured as a two-dimensional or three-dimensional flat panel display that can present endoscopic images to the physician without a need for the physician to insert their head into a viewer housing.

Physician console 120 also includes a pair of input devices including a left human interface device (HID) and a right HID, configured to be manipulated by the physician's left and right hands, respectively. Each of the HIDs can include a handle and/or finger inputs that are manipulated by a user's hands to control a corresponding instrument and/or corresponding robotic manipulator. For example, the left HID 227L may be controlled by a user's left hand to control a left-hand instrument manipulated by a first robotic arm of the surgical robot, and the right HID 227R may be controlled by a user's right hand to control a right-hand instrument manipulated by a second robotic arm of the surgical robot. In the illustrated example, each of the HIDs physically supported by an armrest 226 of the physician console 120 and/or the pillar 226 by a respective positioning arm, including a left positioning arm 228L and a right positioning arm 228R. Such positioning arms can include a series of links and series of joints, including a gimbal-based support, that supports the respective HID in space while permits the respective HID to be manipulated in six degrees of freedom to control a corresponding position (e.g., location and/or orientation) of the respective instrument. Alternatively, or in combination, each of the left HID or right HID can include graspers or buttons that may be actuated by the user's respective hands to actuate the instrument (e.g., to open or close instrument jaws). Although grounded, gimbal-based HIDs are shown, in various configurations, the physician console may employ in grounded (e.g., free-floating or wireless HIDs), or any suitable input devices for the HIDs, without departing from principles described herein.

As seen in FIG. 2, physician console 120 can also include comprising a footboard 231 (or foot pedal assembly) having one or more foot pedals 233. The foot pedal(s) 233 may be coupled to or otherwise positioned at the base 221 of the physician console and may be actuated by a user's feed to control various functionality of the system. For example, in some configurations, various foot pedals may be used to perform ancillary functions of the system, such as activating energy delivery, switching control of instruments, clutch instruments, or firing a staple, for example.

In the illustrated example, physician console 120 includes an additional display, shown in FIG. 2 as a touchscreen display 225 positioned on the armrest 226. The touch screen display 225 can provide an additional interface for a physician to interact with the system. For example, the touchscreen display 225 can provide an additional output interface for displaying various setting or status information associated with the surgical system 100, including hand assignment information associated with the various instruments or robotic arms controlled by the left or right HIDs. Alternatively, or in combination, the touchscreen display 225 can provide an input interface for controlling system settings or controlling hand assignment for various instruments, as further described herein.

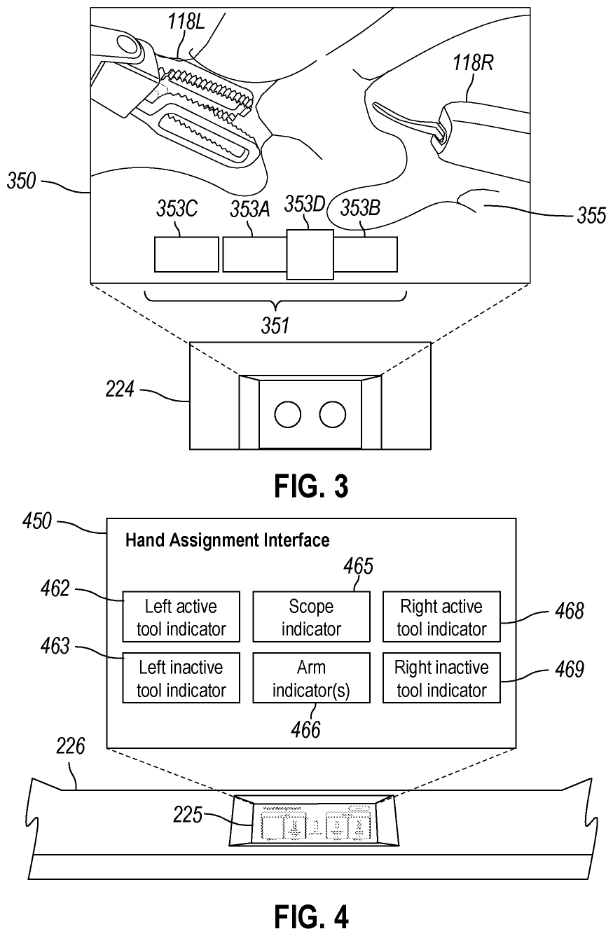

FIGS. 3-4 depict examples of graphical user interfaces that may be employed in the physician console 120, in accordance with some embodiments. FIG. 3 depicts an example of a viewer interface that may be employed in viewer display 224, and FIG. 4 depicts an example of a hand assignment interface that may be employed in touchscreen display 225. In some configurations, these interfaces may be configured to be dynamically updated in accordance with each other.

As seen in FIG. 3, the viewer display 224 can be configured to display a viewer interface 350. The viewer interface 350 can include a graphical user interface (GUI) that includes an endoscopic image 355, as well as a viewer overlay 351 (sometimes referred to herein as “a surgeon overlay user interface (UI)”) over or in concert with the endoscopic image 355. The endoscopic image 355 can correspond to, for example, an image or series of images/video captured by an endoscope of the surgical system (e.g., a laparoscope). The endoscopic image 355 can include an anatomical site of a patient and instruments controllable by the physician console via surgical robot, allowing the physician to control the instruments while observing a real-time image feed of those instruments as captured by the endoscope. In the illustrated configuration, the endoscopic image 355 includes a left actively controlled instrument 118L, which can correspond to an instrument actively controlled by a left hand input device of the physician console (e.g., left HID 227L of FIG. 2), and a right actively controlled instrument 118R, which can correspond to an instrument controlled by a right hand input device of the physician console (e.g., right HID 227R of FIG. 2).

The viewer overlay 351 of the viewer interface 350 can further display various information associated with the surgical system, including information associated with the instruments controlled or controllable by the system. For example, the viewer overlay 351 can display various icons 353A-D (or “windows”) that can each indicate instrument information associated with a corresponding instrument controlled or controllable by the system (e.g., identifying the type of instrument and/or function associated with the instrument). As seen in FIG. 3, a first icon 353A can indicate the left actively controlled tool 118L, and a second icon 353B can indicate the right actively controlled tool 118R. Because the surgical system may involve more than two instruments or arms controllable by the robot, the viewer overlay 351 can additionally display a third icon 353C indicating an inactive instrument that is assigned for left hand control but is not currently actively controlled. The viewer overlay 351 can additionally display a scope indicator 353D, which as noted above may be controllable by both hands or may be otherwise not assigned to a specific hand.

As noted above, it may be beneficial to provide a capability for the physician or user to assign or reassign various instruments or hand assignments. FIG. 4 depicts an example of a hand assignment interface 450, which can be displayed on a touchscreen display 225 in an armrest 226 of the physician console. In various configurations, the touchscreen display 225 may provide a convenient and user friendly interface for the physician to observe and/or updated hand assignments without requiring such updates to be made through the primary viewer display. In some configurations, the hand assignment interface 450 can be surfaced via one of many access points located on the touchscreen user interface (UI). In some configurations, the hand assignment interface 450 or information in the touch screen display 225 can be directly linked to the viewer interface 350 (e.g., the information in the viewer overlay 351 of FIG. 3). For example, updates made to the viewer interface, such as making an instrument active or inactive using foot pedals of the physician console, can be dynamically reflected in the hand assignment interface. As another example, updates made to the hand assignment interface, such as switching an instrument from control of one hand to the other hand, can be dynamically reflected in the viewer interface.

FIG. 4 depicts various elements of the hand assignment interface 450 that can be included in various configurations. As seen in FIG. 4, the hand assignment interface 450 can include various indicators indicating or identifying which tools are associated with various active or inactive slots of the left or right hand input devices. For example, as seen in FIG. 4, the hand assignment interface 450 can include a left hand indicators 462, 463 indicating tools that are controlled by, or assigned to, the left hand input device, including a left active tool indicator 462 indicating which tool (if any) is actively controlled by the left hand input device and a left inactive tool indicator 463 indicating which tool (if any) is assigned to the left hand input device but is not under active control by that input device. The hand assignment interface 450 can also include a right hand indicators 468, 469 indicating tools that are controlled by, or assigned to, the right hand input device, including a right active tool indicator 468 indicating which tool (if any) is actively controlled by the right hand input device and a right inactive tool indicator 469 indicating which tool (if any) is assigned to the right hand input device but is not under active control by that input device. In some instances, the various tool indicators may include, for example, one or more of a name of the type of instrument, an image or rendering of the type of instrument, and/or an identification of a function of the instrument.

In some configurations, the hand assignment interface 450 can further include a scope indicator 465, which may indicate an endoscope coupled to the robot, controlled by the surgeon console, or otherwise in use by the surgical system. In some instances, the scope indicator 465 may be presented as a central instrument or may be presented independently from the left or right hand tool indications to reinforce, visually, that the scope is not assigned to a particular one of the left or right hand input devices. In some instances, the scope indicator 465 can identify a type of scope and/or status information of the scope. For example, the scope indicator can identify whether the endoscope in use by the surgical system is a zero-degree laparoscope, a thirty-degree laparoscope, or another type of endoscope. Alternatively, or in combination, the scope indicator 465 may indicate a current orientation of the scope.

In some configurations, as seen in FIG. 4, the hand assignment interface 450 can further include one or more arm indicators 466 indicating which of several robotic arms is coupled with or controlling each tool. These may include, for example, arm indicators associated with each of the left or right hand tools. Alternatively, or in combination, these arm indicators 466 may include indication of which arm is controlling the scope. In some instances, each arm is identified by an appropriate coding scheme to discriminate the various arms, such as numerical (e.g., arm 1, arm 2, arm 3, etc.), alphabetical (e.g., arm A, arm B, arm C, etc.), or color coded (e.g., blue arm, green arm, purple arm, etc.). In some configurations, as instruments are loaded, removed, or exchanged on various robotic arms, various elements of the hand assignment interface 450 can be configured to dynamically update to reflect the current system or robot status.

The hand assignment interface 450 can include various visual and/or interactive GUI elements to provide a user friendly or appealing interface. Examples of hand assignment interfaces are further described below, including handedness-centric interfaces, arm-centric interfaces, card-based interfaces, and robot model-based interfaces, each of which may include various features to reinforce certain information and/or provide instinctive interactions. In some variations, features from the various examples may be combined or modified in different ways without departing from the concepts described herein.

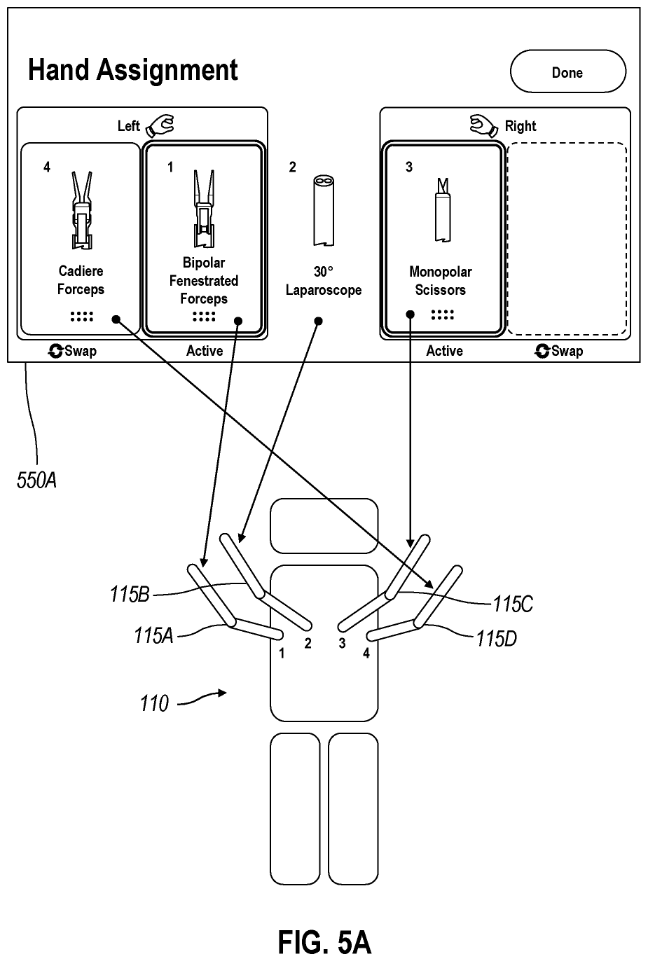

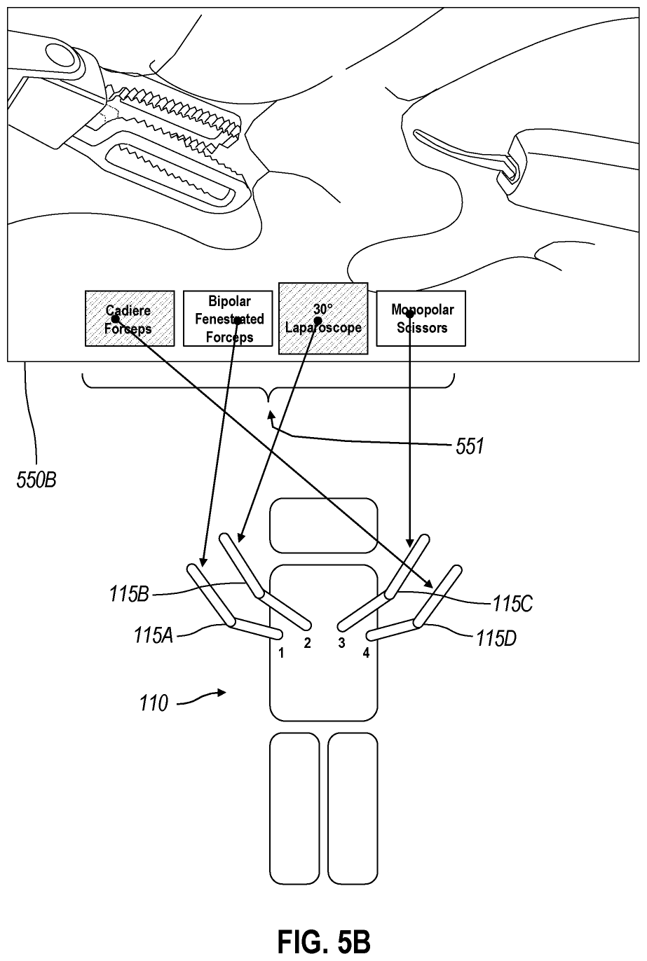

FIGS. 5A-5B depict examples of interfaces employing a handedness-centric approach, in accordance with some embodiments. FIG. 5A depicts an example of a hand assignment interface 550A employing a handedness-centric approach, and FIG. 5B depicts an example of a viewer interface 550B employing a handedness-centric approach and linked to the hand assignment interface 550A of FIG. 5A. FIGS. 5A-5B also depict a schematic representation of surgical robot 110, to illustrate certain concepts associated with a handedness-centric approach.

As seen in FIGS. 5A-5B, the hand assignment interface 550A can present an indication of which hand various instruments are assigned to, as well as the corresponding robotic arms. The handedness-centric approach to the hand assignment interface 550A can present the various instrument and hand indications in an arrangement that emphasizes the left or right hands, independent of the physical arrangement of the robotic arms. Here, the instruments controlled or assigned to the left hand input device (denoted by the slots under the “left” indicator) are generally positioned on the left portion of the interface, while instruments controlled or assigned to the right hand input device (denoted by the slots under the “right” indicator) are generally positioned on the right portion of the interface. This interface may be presented independently of the arrangement of robotic arms in physical space, which may be arbitrarily arranged independently of the handedness of each instrument.

In this example, the surgical robot includes four robotic arms 115A-D, each numbered 1-4, respectively, by arm indicators in the UI. Each robotic arm is also controlling a corresponding instrument, such that four instruments are controlled by these four robotic arms. In the illustrated example, robotic arm 115A (identified as arm 1) is controlling bipolar fenestrated forceps, robotic arm 115B (identified as arm 2) is controlling the laparoscope, robotic arm 115C (identified as arm 3) is controlling monopolar scissors, robotic arm 115D (identified as arm 4) is controlling cadiere forceps. Each of these instruments and arms is identified by corresponding indicators positioned on a UI element associated with the corresponding instrument/arms (e.g., a numerical indication of the corresponding arm, and the name and image of the corresponding instrument in this example). This interface can thus communicate various information to the physician, including what tool is active on their left hand, what tool is active on their right hand, what hand their inactive tool is assigned to, what hand has an open inactive slot, and the arm each tool is loaded on. In this instance, only one hand can only control, or be assigned to, up to two instruments, so this interface can also reinforce that restriction visually.

The hand assignment interface 550A shown in FIG. 5A depicts a card-based interface that a user may interact with to reassign instruments, as further described below. It is noted that this interface may reassign which input device (e.g., left or right HID) is controlling the instrument without affecting or moving the physical instruments or robotic arms. Here, each assignable instrument is positioned on a card corresponding to that instrument (and the robotic arm to which that instrument is mounted). A user may interact with these cards to move various cards to various slots, as appropriate, in order to reassign a handedness of a corresponding instrument. To reinforce visually that the laparoscope cannot be assigned to either hand, the laparoscope element (and its associated indicators) is placed in the center of the interface and not positioned on a card.

FIG. 5B depicts a viewer interface 550B that can be displayed in concert with the hand assignment interface 550A, and that may be linked to the information in the hand assignment interface. Here, the viewer interface 550B also utilizes a handedness-centric approach that corresponds to the handedness-centric approach of FIG. 5A. The viewer interface 550B includes a viewer overlay 551 having an arrangement of icons corresponding to the various instruments controlled by the robot 110. For clarity, only instrument names are depicted in the viewer overlay 551, but the various instrument icons may include any of the same information depicted in the hand assignment interface (e.g., the cards or the laparoscope UI element) and/or may include additional information. Here, the arrangement of the instrument icons matches the arrangement of the instrument cards and laparoscope UI element shown in FIG. 5A, with the left portion of the interface displaying instruments controlled by the left hand input device, a right portion of the interface displaying instruments controlled by the right hand input device, and the scope icon presented in the center of the interface.

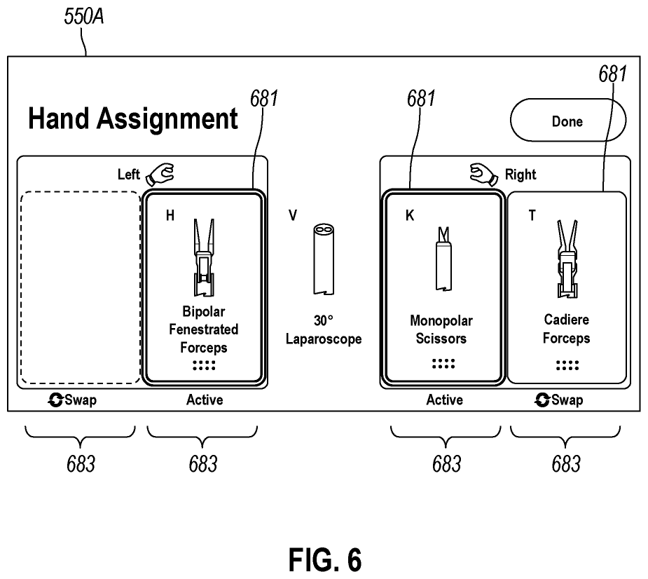

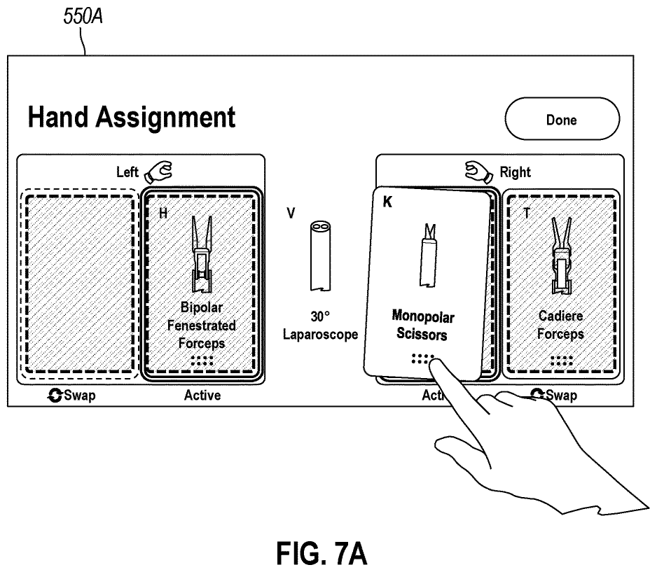

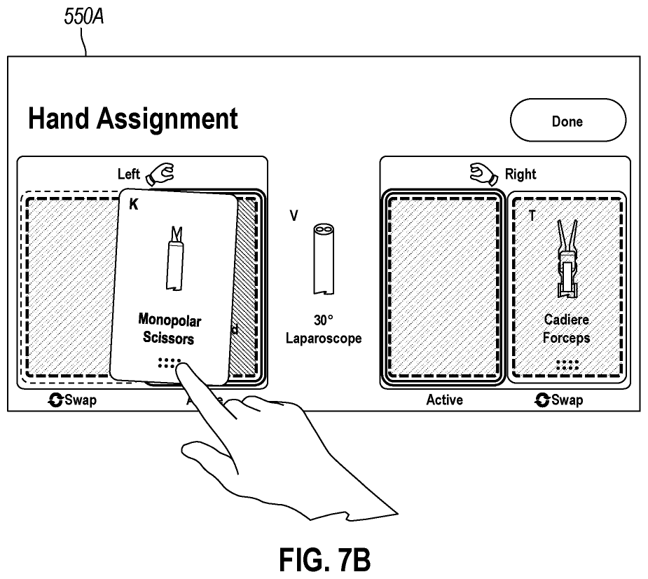

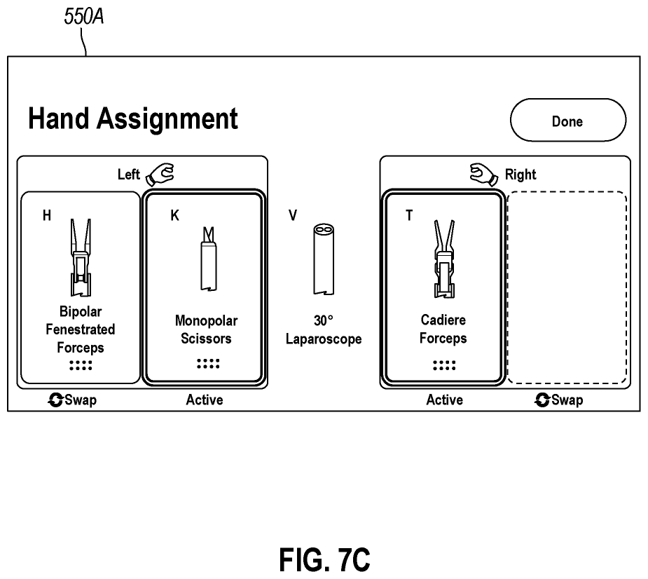

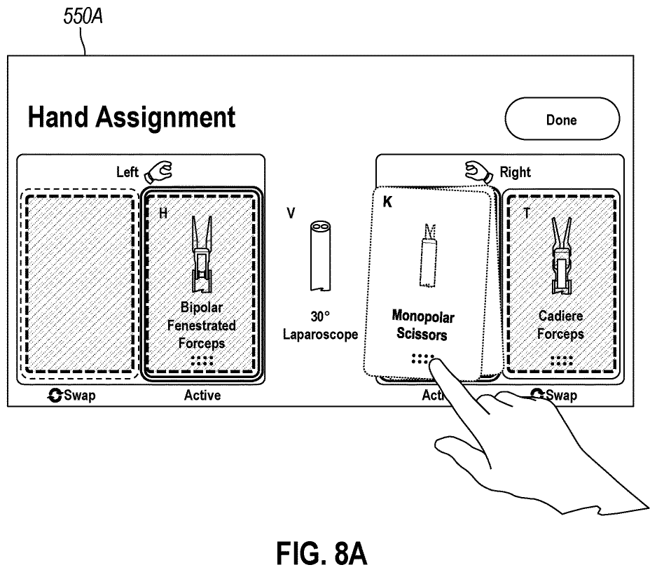

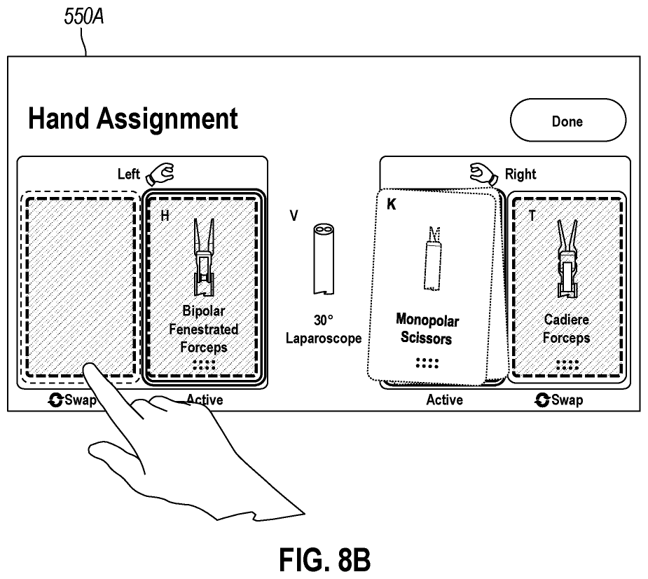

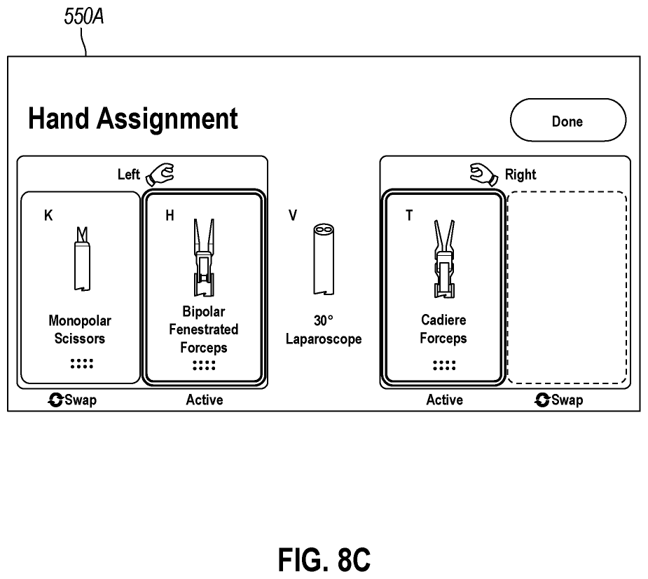

FIG. 6 depicts the hand assignment interface 550A employing a card-based approach to the handedness-centric interface. The hand assignment interface includes a plurality of cards 681, each corresponding to an instrument coupled to a corresponding robotic arm (e.g., H, K, T), and a plurality of card slots 683, each corresponding to an assignable slot. Here, the swap slot under the left hand icon corresponds to a left hand inactive tool, the active slot under the left hand icon corresponds to a left hand actively controlled tool, swap slot under the right hand icon corresponds to a right hand inactive tool, the active slot under the right hand icon corresponds to a right hand actively controlled tool. The system may include various interaction schemes permit a user (e.g., physician) to reassign instruments to various slots. Two such interactions are described below, including a drag and drop interaction (FIGS. 7A-7C) and a multi-tap interaction (FIGS. 8A-8C) with the respective cards and slots.

Due to the number of scenarios possible with hand assignment, there may be restrictions in place for which spots a tool can be moved to. In various schemes, the hand assignment interface 550A may dynamically update in response to these user interactions, and may include various dynamic visual elements, such as highlighting, borders, or coloring, to communicate information to the user, such as permissible reassignment slots or restrictions on hand reassignments.

FIGS. 7A-7C depict an example of a drag and drop interaction with the card-based interface of FIG. 6, in accordance with some embodiments. As illustrated, if a user wants to assign an instrument to a different hand, they can drag a card and move it to a different location or slot.

FIG. 7A depicts the drag and drop interaction in a first state where a user presses and holds a card to be reassigned (in this instance, monopolar scissors). Upon the user interaction beginning to drag the card (e.g., pressing, holding, and moving the card), available drop targets may begin to pulse (e.g., with a white dashed line border and background). This is depicted schematically in FIG. 7A via the sharing and dashed line border shown for all of the left and right active and inactive slots, as all the slots are acceptable drop targets in this example.

FIG. 7B depicts the drag and drop interaction in a second state, after the user has dragged the card to a desired slot (left active slot in this example). When the card is held over the acceptable drop target, the visualization associated with the slot may again change, for example, by changing the color of the background and border from white to teal. This visually indicates which slot the card will go into if the card is released in that moment.

FIG. 7C depicts the drag and drop interaction in a third state, after the user has released the card in the desired slot. Once the card is released to an acceptable location, one or more of the other instruments may be reassigned. In this example, once the monopolar scissors are released into the left active slot, the bipolard fenestrated forceps automatically move to the left inactive slot since they had previously occupied the left active slot. Additionally, since the right active slot, where the monopolar scissors previously were, was vacated, the cadiere forceps automatically move from the right inactive slot into the right active slot.

FIGS. 8A-8C depict an example of a tap-based interaction with the card-based interface of FIG. 6, in accordance with some embodiments. The same automatic reassignments and/or restrictions may be present in this example as in FIGS. 7A-7C, with the difference being that the user interaction may involve multiple taps as opposed to a drag and drop, and the visualizations on the hand assignment interface 550A may adjust accordingly.

FIG. 8A depicts an example of the tap-based interaction in a first state, where a user has tapped a card. Upon tapping the card, the tapped card may begin to shake or may otherwise be visualized differently to indicate that it is ready to be moved. As in the drag and drop example, the acceptable drop targets may be visualized differently, for example by pulsing white.

FIG. 8B depicts the tap interaction in a second state, where the user has tapped a desired slot for the card. After the initial tap from FIG. 8A, the user can then tap any of the acceptable drop targets to move the card to the new slot. In this example, the user selects the left inactive slot.

FIG. 8C depicts the tap-based interaction in a third state, after the user has tapped the desired slot for the card. Here, the user has selected the left inactive slot for the instrument monopolar scissors instrument card, so the bipolar fenestrated forceps remain in the left active slot. However, as with the drag and drop example, the cadiere forceps move to the right active slot as it is the only instrument remaining assigned to the right hand after the interaction.

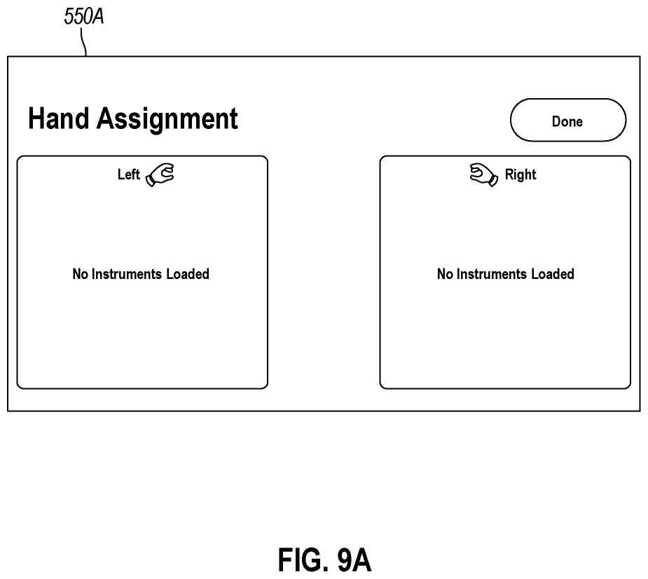

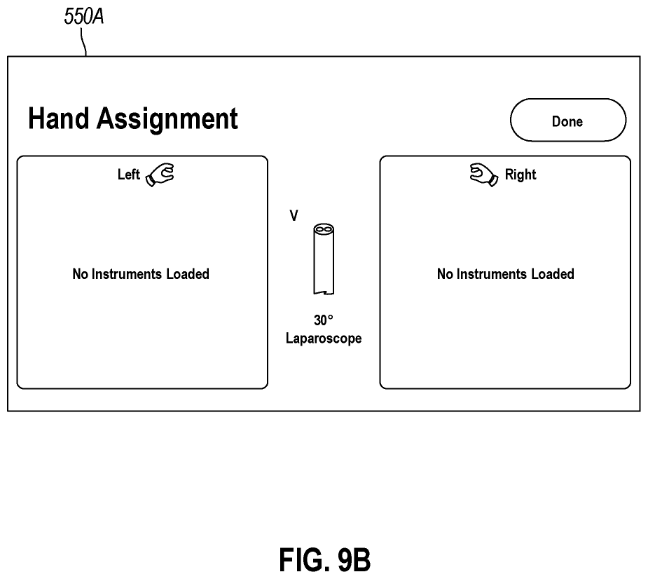

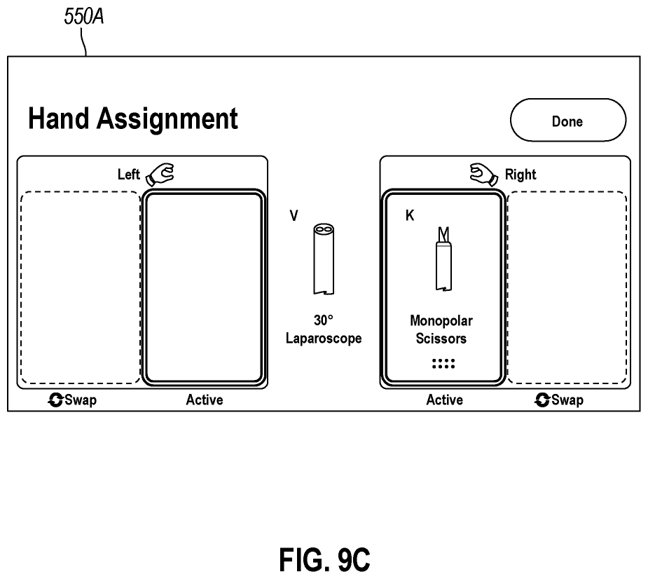

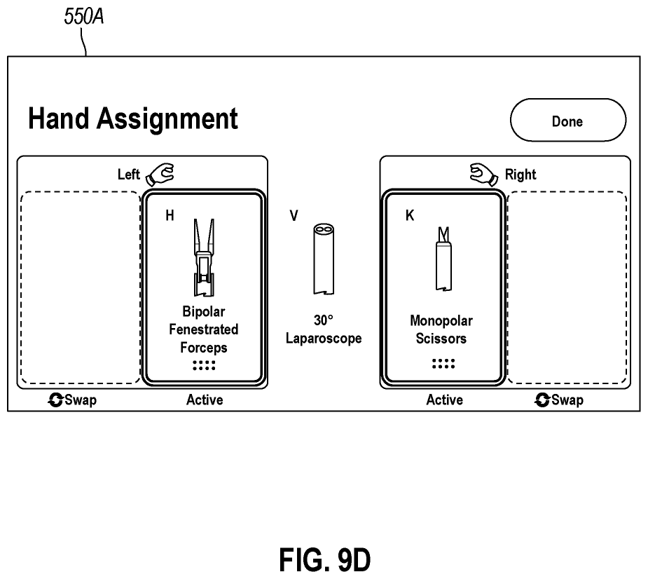

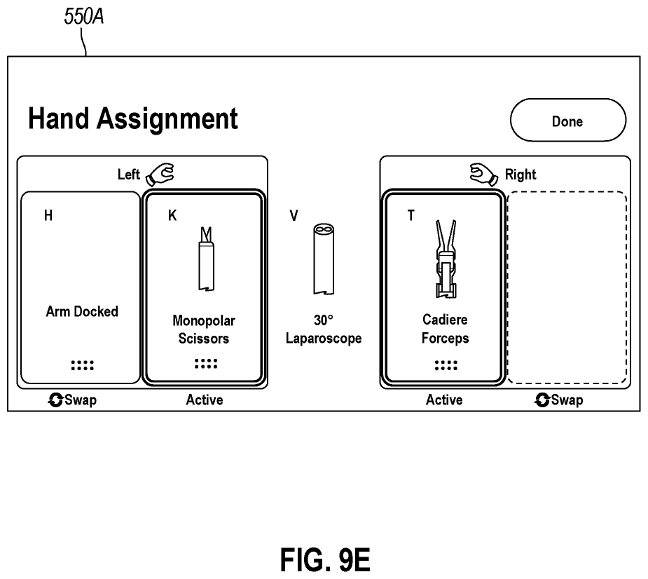

In some configurations, the hand assignment interface can dynamically update based on the state of the robot. FIGS. 9A-9E depict various examples of instances of the hand assignment interface 550A that may be employed, during an initial set up interaction, to reflect a state of the robot. In various configurations, the robot can be configured to automatically detect the various states using any of the sensing or detection schemes noted above.

FIG. 9A depicts the hand assignment interface 550A in a first state, where no instruments are loaded. As illustrated, the left and right hand instrument slots may be empty, and may further provide a text-based or other type of indication to notify the user that no instruments are loaded to the robot.

FIG. 9B depicts the hand assignment interface 550A in a second state, where a scope is loaded. As illustrated, the center portion of the interface may dynamically update to show the scope UI element, along with an identification of information about the scope, such as a type of scope (e.g., 30 degree laparoscope in this example), or an arm upon which the scope is mounted (e.g., arm V in this example).

FIG. 9C depicts the hand assignment interface 550A in a third state, where two instruments are loaded. Here, the appropriate instrument card shows up to illustrate what type of instrument has been mounted, as well as which arm the instrument is mounted to. The interface may also automatically determine a hand assignment at this stage, based on an initial algorithmic determination. This initial hand assignment may be later changed by the user interacting with the hand assignment interface, as discussed previously.

FIG. 9D depicts the hand assignment interface 550A in a fourth state, where three instruments are loaded. Again, the appropriate instrument card shows up to illustrate what type of instrument has been mounted, as well as which arm the instrument is mounted to.

FIG. 9E depicts the hand assignment interface 550A in a fifth state, where three instruments are loaded and a fourth instrument is removed from an arm that is docked to its respective cannula. Here, a card is presented to show which arm has been docked, and an indication that the arm is docked without a corresponding instrument being mounted thereto.

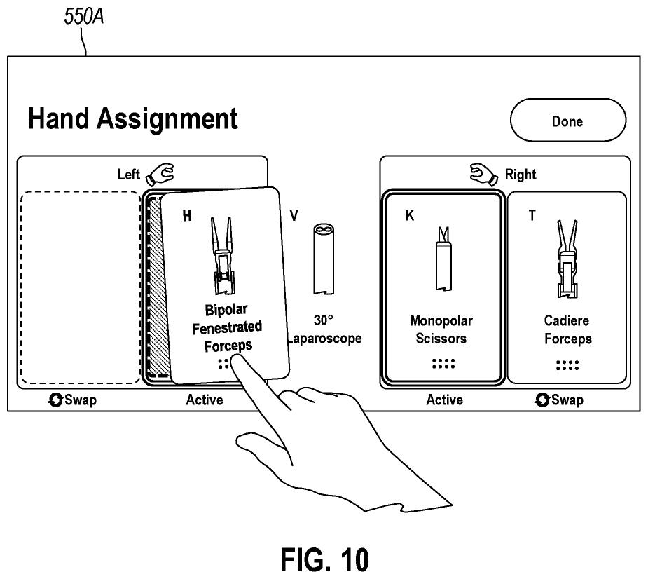

In some instances, the hand assignment interface may enforce restrictions against certain instrument reassignments, e.g., for safety, usability, or other considerations. FIG. 10 depicts an example of a hand assignment interface when a user attempts to reassign an instrument to a hand that is already full. Here, none of the other cards show up as acceptable drop targets, as the system may prevent reassignment to an arm that is already full.

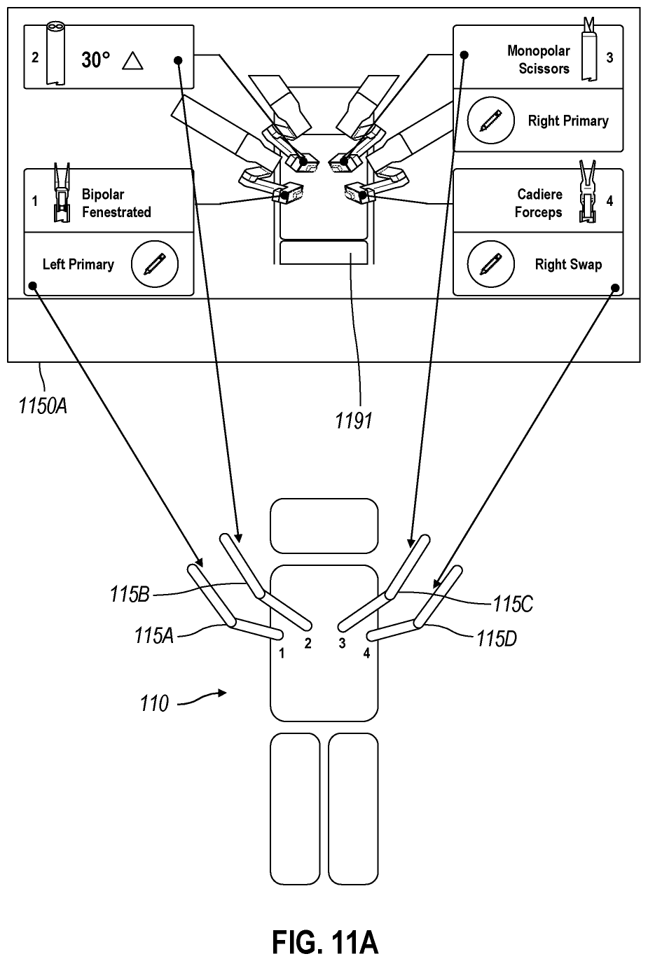

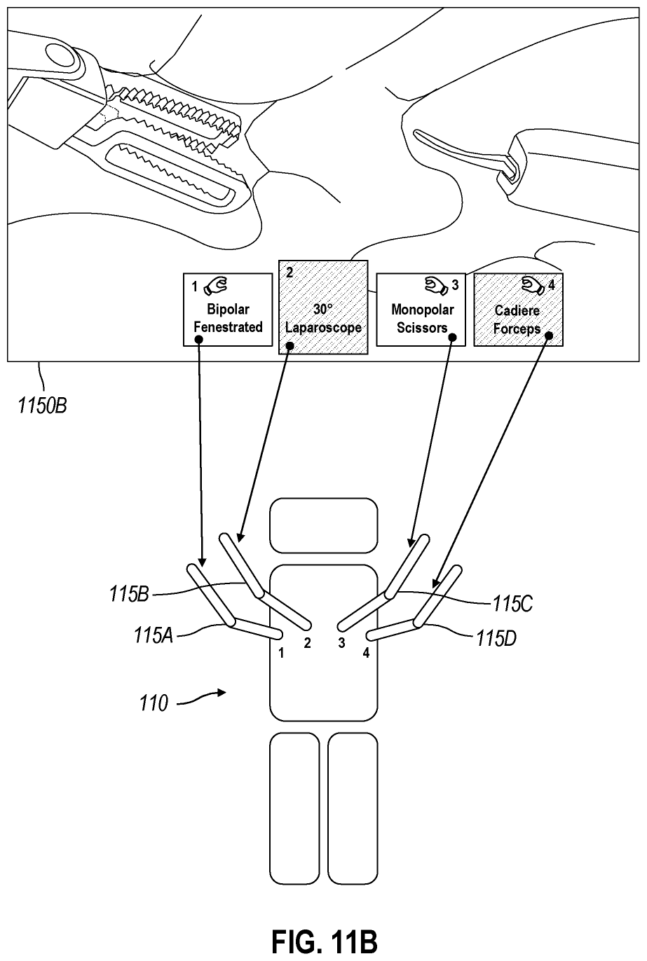

FIGS. 11A-11B depict examples of interfaces employing an arm-centric approach, in accordance with some embodiments. FIG. 11A depicts an example of a hand assignment interface employing the arm-centric approach with a robot model, and FIG. 11B depicts an example of a viewer interface employing the arm-centric approach in accordance with the hand assignment interface of FIG. 11A.

As seen in FIGS. 11A-11B, the hand assignment interface 1150A can present an indication of which hand various instruments are assigned to, as well as the corresponding robotic arms. The arm-centric approach to the hand assignment interface 1150A can present the various instrument and hand indications in an arrangement that emphasizes the arrangement of robotic arms 115A-D, independent of the handedness of each arm or instrument. Here, the instruments controlled or assigned to the left oriented arms are generally positioned on the left portion of the interface, while instruments controlled or assigned to the right oriented arms are generally positioned on the right portion of the interface. This interface may be presented independently of the handedness of each arm.

In this example, the surgical robot includes four robotic arms 115A-D, each numbered 1-4, respectively, by arm indicators in the UI. Each robotic arm is also controlling a corresponding instrument, such that four instruments are controlled by these four robotic arms. In the illustrated example, robotic arm 115A (identified as arm 1) is controlling bipolar fenestrated forceps, robotic arm 115B (identified as arm 2) is controlling the laparoscope, robotic arm 115C (identified as arm 3) is controlling monopolar scissors, robotic arm 115D (identified as arm 4) is controlling cadiere forceps. Each of these instruments and arms is identified by corresponding indicators positioned on a graphical model 1191 of the robot, in a location corresponding to the corresponding arm the instrument is assigned to. This interface can thus communicate similar information as discussed in the handedness-centric approach, but with an arm-centric orientation to emphasis situational awareness with respect to the robot. The model 1191 can be, for example, a three-dimensional graphical model of the robot that is dynamically updated based on the state of the robot 110. Alternatively, or in combination, the model can include a two-dimensional model or other graphical representation that is representative of the robotic arms and their relative orientations.

The hand assignment interface 1150 shown in FIG. 11A depicts a menu-based interface that a user may interact with to reassign instruments. The underlying logic associated with reassigning instruments or handedness of instrument may be similar to that described above with respect to the handedness-centric approach.

FIG. 11B depicts a viewer interface 1150B that can be displayed in concert with the hand assignment interface 1150A, and that may be linked to the information in the hand assignment interface. Here, the viewer interface 1150B also utilizes an arm-centric approach that corresponds to the arm-centric approach of FIG. 11A. The viewer interface 1150B includes a viewer overlay having an arrangement of icons corresponding to the various instruments controlled by the robot 110. Here, the viewer overlay employs an arm-centric approach linked to the hand assignment interface shown in FIG. 11A.

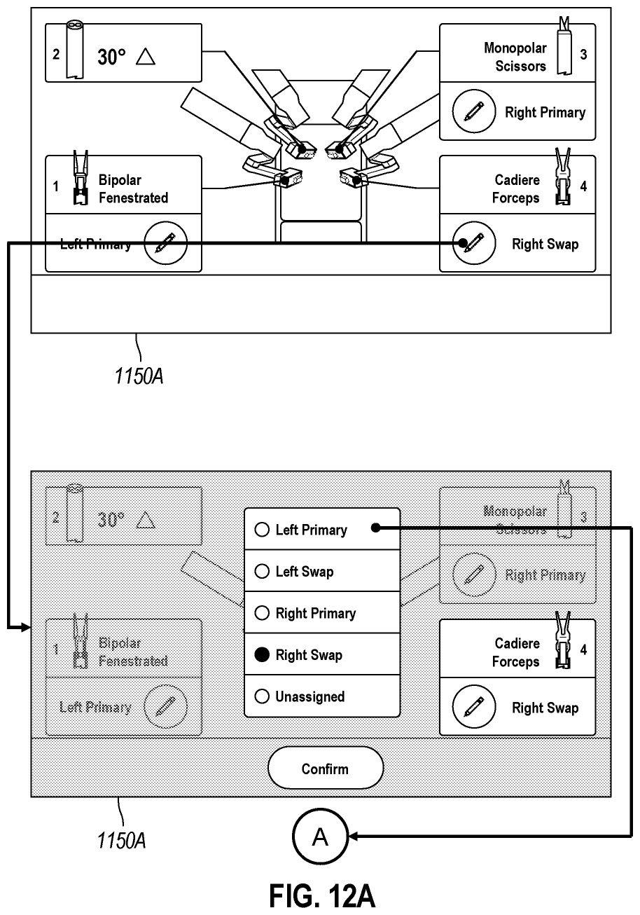

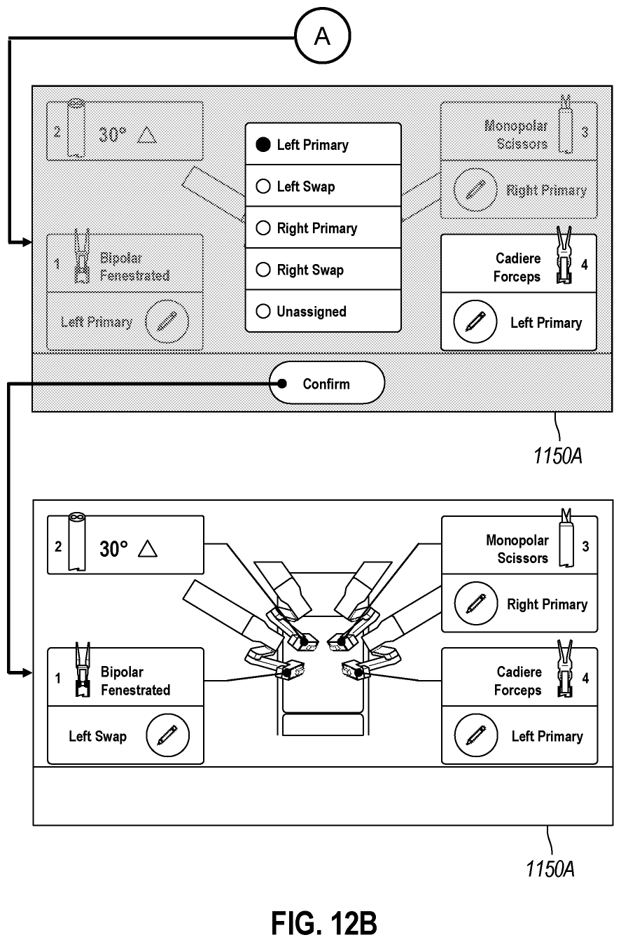

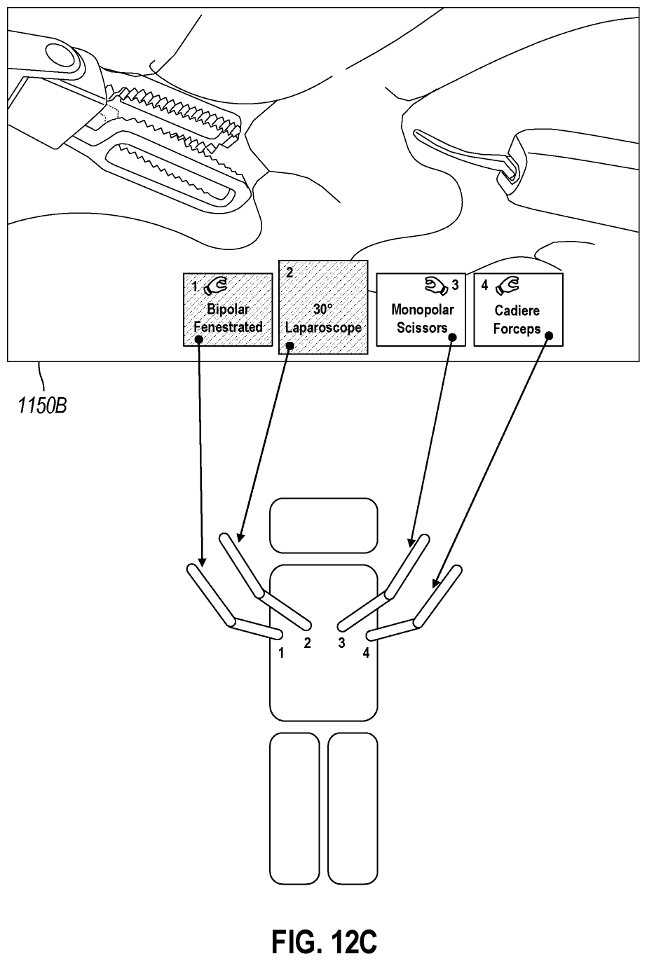

FIGS. 12A-12B depict a method of interacting with the hand assignment interface 1150A, in accordance with some embodiments. FIG. 12A depicts the hand assignment interface at a first step, where an instrument is selected for reassignment by a user, and at a second step, where an assignment selection menu is presented to the user. FIG. 12B depicts the hand assignment interface at a third step, after the user has selected a desired assignment for the instrument, and at a fourth step, after the user has confirmed the instrument reassignment. As with the handedness-centric approach, the instruments may be automatically reassigned in accordance with intelligent logic.

FIG. 12C depicts viewer interface 1150B after the reassignment. As with the earlier example, the viewer interface may be linked and may be dynamically updated in accordance with the hand assignment interface.

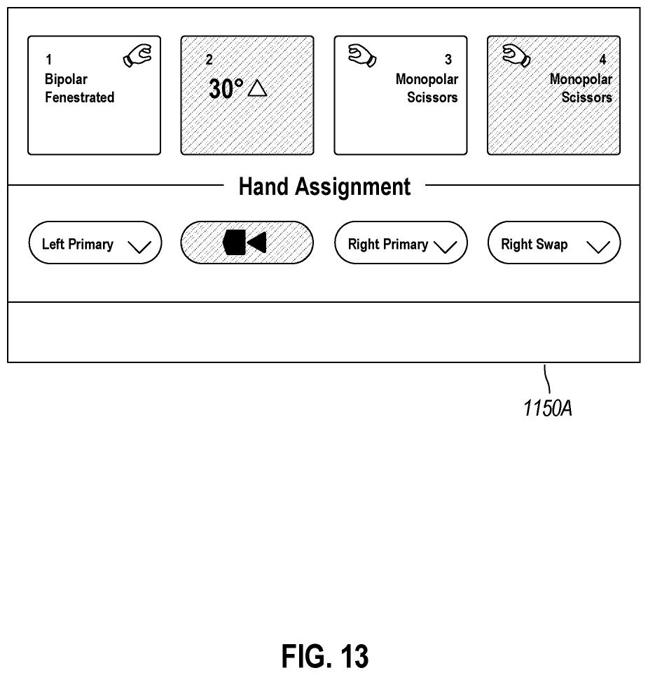

FIG. 13 depicts an example of a hand assignment interface 1350A employing an arm-centric approach without a robot model, in accordance with some embodiments.

Various examples disclosed herein described implementation of a hand assignment interface on a touchscreen of an armrest on a physician console. However, it will be appreciated that, in some variations, the hand assignment interfaces described herein can be presented in any suitable display in a surgical system. For example, the hand assignment interfaces described herein can be presented on various touchscreens or non-touch sensitive displays. Further, in some variations, hand assignment interfaces can be updated through touch sensitive interactions or various other types of interactions, such as a mouse pointer interactions, scroll wheels, or the like.

Various examples disclosed herein describe usage of the surgical system to perform a procedure on a patient, wherein instruments are inserted into a body of the patient. In various configurations, the surgical system may be used, for instance, in educational or lab settings, where a body portion of a model, cadaver, animal, or inanimate object is placed upon the headrest. Such methods may be useful for surgeon training, product testing, development applications, or the like. Accordingly, it will be understood that methods described herein are not limited to medical procedures performed on a human body but can be implemented on bodies or objects that are not part of a live patient or human.

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

Example Combination 1: A robotic surgical system may include: a left hand input device; a right hand input device; a display; and a processor configured to: present, on the display, a graphical user interface for assigning instruments to the left hand input device or the right hand input device; and, upon a user interaction that assigns a first instrument to the left hand input device or the right hand input device, reassign a second instrument.

Example Combination 2: The robotic surgical system of Example Combination 1, where the graphical user interface may include a handedness-centric overlay, where a left portion of the handedness-centric overlay displays an instrument assignment for the left hand input device and a right portion of the handedness-centric overlay displays an instrument assignment for the right hand input device.

Example Combination 3: The robotic surgical system of Example Combination 1 or Example Combination 2, where the graphical user interface may include an arm-centric overlay, where a left portion of the arm-centric overlay displays an instrument assignment for a left-oriented robotic arm and a right portion of the arm-centric overlay displays an instrument assignment for a right-oriented robotic arm.

Example Combination 4: The robotic surgical system of any of any one of Example Combinations 1-3, where the display is a touchscreen display positioned on an armrest of a physician console.

Example Combination 5: The robotic surgical system of any of any one of Example Combinations 1-4, where, upon the user interaction assigning the first instrument to an active slot of the left hand input device when the second instrument is in the active slot of the left hand input device and an inactive slot of the left hand input device is unassigned, the processor is configured to reassign the second instrument to the inactive slot of the left hand input device.

Example Combination 6: The robotic surgical system of any of any one of Example Combinations 1-5, where the processor is configured to prevent assignment of an instrument to the left hand input device when the left hand input device is fully assigned.

Example Combination 7: The robotic surgical system of any of any one of Example Combinations 1-6, where the processor is configured to dynamically update the graphical user interface based on a state of a robot controlled by the left and right hand input devices.

Example Combination 8: The robotic surgical system of any of any one of Example Combinations 1-7, where: the graphical user interface may include a plurality of cards and a plurality of slots; each of the cards corresponds to an instrument; and each of the slots corresponds to an instrument assignment associated with the left hand input device or the right hand input device.

Example Combination 9: The robotic surgical system of any one of Example Combinations 1-8, where the plurality of slots include: a left hand active slot corresponding to an active instrument controlled by the left hand input device; a right hand active slot corresponding to an active instrument controlled by the right hand input device; a left hand inactive slot corresponding to an inactive instrument assigned to the left hand input device; and a right hand inactive slot corresponding to an inactive instrument assigned to the right hand input device.

Example Combination 10: The robotic surgical system of any one of Example Combinations 1-9 or 10, where each of the cards includes a robotic arm identifier and an instrument identifier.

Example Combination 11: The robotic surgical system of any of any one of Example Combinations 1-10, where each of the cards is assignable to a valid slot of the plurality of slots via a drag and drop user interaction involving dragging the card being reassigned to the valid slot and releasing the card in the valid slot.

Example Combination 12: The robotic surgical system of any of any one of Example Combinations 1-11, where each of the cards is assignable to a valid slot of the plurality of slots via a multi-tap interaction involving a first tap to the card being reassigned and a second tap to the valid slot.

Example Combination 13: The robotic surgical system of any of any one of Example Combinations 1-12, where the graphical user interface may include: a graphical model of a plurality of robotic arms; and a hand assignment identifier associated with each of the robotic arms.

Example Combination 14: The robotic surgical system of any of any one of Example Combinations 1-13, where the display is a touch screen display, and the robotic surgical system further may include a viewer display configured to display an endoscopic image of instruments controlled by the left or right hand input device, where the processor is configured to present, on the viewer display, an overlay that is linked to the a graphical user interface for assigning instruments.

Example Combination 15: The robotic surgical system of claim any of claims 1-5, where, upon the user interaction assigning the first instrument from an active slot of the right hand input device when the second instrument is in the inactive slot of the right hand input device, the processor is configured to reassign the second instrument to the active slot of the right hand input device.

Use of “or” is intended in the inclusive rather than exclusive sense, unless explicitly stated otherwise or the context clearly dictates otherwise. Thus, for example, reference to “A” or “B” can encompass “A” only, “B” only, or both “A” and “B.” As another example, reference to “A, B, or C” can encompass “A” only, “B” only, “C” only, or any combination of two or more of “A” or “B” or “C.” Accordingly, the term “or” should be generally understood as equivalent to “and/or” unless stated otherwise or the context clearly dictates to the contrary.

It should be appreciated that any specific order of steps shown or described herein is illustrative in nature and should not be construed as required unless explicitly stated or the context clearly dictates otherwise. Thus, for example, with respect to any processes or methods herein, any two or more steps or stages in a method or process may performed serially or in parallel, in any combination, and may be performed in any order, unless explicitly stated or the context clearly dictates otherwise.

In some instances, relative positions or orientations are used, such as top, bottom, upper, lower, forward, backward, front, rear, left, right, up down, horizontal, vertical, longitudinal, lateral, or the like. These terms may be used to refer to an arbitrary frame of reference or a frame of reference shown in the drawings, for purposes of explanation or to demonstrate the relative spatial configurations associated with various elements. These terms should not be understood to require any particular gravitational or other frame of reference unless explicitly stated or the context clearly dictates otherwise.

To the extent any headings are used through this description, these headings are used for convenience only and should not be construed as limit the scope of disclosure or the description under a heading to only the topic associated with the heading in anyway.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various examples, configurations, or embodiments of the present technology, further adaptations of the systems or methods described herein may be accomplished by appropriate modifications by one of ordinary skill in the art without departing from the scope of the technology described herein. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the claimed subject matter should be considered in terms of the following claims and is understood not to be limited to the details of structure and operation shown and described in the specification and drawings.

1. A robotic surgical system comprising:
a left hand input device;
a right hand input device;
a display; and
a processor configured to:
present, on the display, a graphical user interface for assigning instruments to the left hand input device or the right hand input device; and,
upon a user interaction that assigns a first instrument to the left hand input device or the right hand input device, reassign a second instrument.

2. The robotic surgical system of claim 1, wherein the graphical user interface comprises a handedness-centric overlay, where a left portion of the handedness-centric overlay displays an instrument assignment for the left hand input device and a right portion of the handedness-centric overlay displays an instrument assignment for the right hand input device.

3. The robotic surgical system of claim 1, wherein the graphical user interface comprises an arm-centric overlay, where a left portion of the arm-centric overlay displays an instrument assignment for a left-oriented robotic arm and a right portion of the arm-centric overlay displays an instrument assignment for a right-oriented robotic arm.

4. The robotic surgical system of claim 1, wherein the display is a touchscreen display positioned on an armrest of a physician console.

5. The robotic surgical system of claim 1, wherein, upon the user interaction assigning the first instrument to an active slot of the left hand input device when the second instrument is in the active slot of the left hand input device and an inactive slot of the left hand input device is unassigned, the processor is configured to reassign the second instrument to the inactive slot of the left hand input device.

6. The robotic surgical system of claim 1, wherein, upon the user interaction assigning the first instrument from an active slot of the right hand input device when the second instrument is in an inactive slot of the right hand input device, the processor is configured to reassign the second instrument to the active slot of the right hand input device.

7. The robotic surgical system of claim 1, wherein the processor is configured to prevent assignment of an instrument to the left hand input device when the left hand input device is fully assigned.

8. The robotic surgical system of claim 1, wherein the processor is configured to dynamically update the graphical user interface based on a state of a robot controlled by the left and right hand input devices.

9. The robotic surgical system of claim 1, wherein:
the graphical user interface comprises a plurality of cards and a plurality of slots;
each of the cards corresponds to an instrument; and
each of the slots corresponds to an instrument assignment associated with the left hand input device or the right hand input device.

10. The robotic surgical system of claim 9, wherein the plurality of slots include:
a left hand active slot corresponding to an active instrument controlled by the left hand input device;
a right hand active slot corresponding to an active instrument controlled by the right hand input device;
a left hand inactive slot corresponding to an inactive instrument assigned to the left hand input device; and
a right hand inactive slot corresponding to an inactive instrument assigned to the right hand input device.

11. The robotic surgical system of claim 9, wherein each of the cards includes a robotic arm identifier and an instrument identifier.

12. The robotic surgical system of claim 9, wherein each of the cards is assignable to a valid slot of the plurality of slots via a drag and drop user interaction involving dragging the card being reassigned to the valid slot and releasing the card in the valid slot.

13. The robotic surgical system of claim 9, wherein each of the cards is assignable to a valid slot of the plurality of slots via a multi-tap interaction involving a first tap to the card being reassigned and a second tap to the valid slot.

14. The robotic surgical system of claim 1, wherein the graphical user interface comprises:
a graphical model of a plurality of robotic arms; and
a hand assignment identifier associated with each of the robotic arms.

15. The robotic surgical system of claim 1, wherein the display is a touch screen display, and the robotic surgical system further comprises a viewer display configured to display an endoscopic image of instruments controlled by the left or right hand input device, wherein the processor is configured to present, on the viewer display, an overlay that is linked to the a graphical user interface for assigning instruments.
